(19) United States
(12) Patent Application Publication
(10) Pub. No.: US 2002/0147454 A1
(43) Pub. Date: Oct. 10, 2002

(54) Building configuration introduced in a surgical-use screw

(76) Inventor: Aziz Rassi Neto, Sao Paulo (BR)

Correspondence Address: LACKENBACH SIEGEL MARZULLO, ARONSON & GREENSPAN, P.C., Penthouse Suite, One Chase Road, Scarsdale, NY, 10583, US

(21) Appl. No.: 10/022,313

(22) Filed: Dec. 17, 2001

(30) Foreign Application Priority Data
Apr. 10, 2001 (BR) MU 8100696-9

(51) Int. Cl. A61B 17/56

(52) U.S. Cl. 606/73

(57) ABSTRACT

A building configuration introduced in a surgical-use screw characterized by the fact of comprising an expandable element (1) formed by a monoblock body (2) with an essentially cylindrical configuration; the expandable element (1) comprises a threaded axial bore (3), which at its distal half (4) undergoes a funneling defining a threaded trunk sector (5); the expandable element (1) plays a set of slots (6), starting from its distal end and reaching as far as approximately half the length measurement of said element (1).

The present report refers to a Utility Model patent dealing with a new building configuration introduced in surgical-use screws, destined to promote the fixation of plates or other elements usually normally used for the human body bone structures recomposition.

We know bone recomposition techniques through the use of mechanical structural elements conventionally comprising the use of metallic pins and/or plates fixed with pins, which actuate both as reinforcement or even as connection elements between damaged or fractured bone structures.

The state of the technique appertaining the present questions comprises the Utility Model patent application MU 7900887-9, of Apr. 16^{th}, 1999, belonging to the applicant hereof, and in the mentioned document MU 7900887-9, it is presented a surgical-use screw basically comprising two modalities, one of them named as expanding and other one named as dilating.

More specifically, as to the expanding type screw, which is indicated by the numerical reference 1, it is verified that it is essentially composed by three basic components, which comprise a spindle 2, which on its anterior end incorporates an expanding terminal 4 defined by a sector with a trunk profile 5, which displays a diameter measurement superior to the general diameter measurement of the spindle 2.

The anterior end 3 of the spindle 2 also counts on a threaded point 6, whose thread 7 reaches the limits of expanding terminal 4.

The body 8 of spindle 2, in its posterior region 9, incorporates a central and axial threaded bore 10, ending at the posterior face 11 of referred spindle 2, a place with two slots 12, destined to receive the tool with which the present surgical screw will be fixated.

The state-of-the-art expanding screw also counts on a cover 13 of essentially tubular shape 14, endowed with longitudinal windows 15 aligned with equally longitudinal slots 16, and cover 13 houses spindle 2, which can be axially moved by means of the rotation of a threaded pin 17 housed in the central and axial threaded bore 10, and the referred pin 17 counts on a pair of rupturable radial pins 18, housed in corresponding indentures 19 on the posterior edge of cover 13, which allows the referred threaded pin 17, when being rotated by means of an appropriate tool, to simultaneously rotate the mentioned cover 13.

The same document MU 7900887-9 also contemplates a variant named as dilating screw, which is indicated by the numerical reference 21, and comprises a dilating element 22, which in its axial bore 23 incorporates a thread sector 24, and the referred dilating element 22 also incorporates a thread sector 25 on its external surface 26.

Despite the undeniable advances of the configuration proposed in patent application MU 7900887-9 in relation to the state-of-the-art considered on the date of its deposit, that is, on Apr. 16^{th}, 1999, the applicant hereof continued his researches, seeking to improve even more the effectiveness of his product by means of changes promoting a higher simplification of its configuration.

Thus, after several studies, the applicant reached a building configuration which is even more efficient than the configuration adopted by the surgical-use screws dealt with in document MU 7900887-9.

In face of the state-of-the-art described above, the present building configuration was developed, introduced in a surgical-use screw, which will be detailedly described with reference to the drawings listed below, in which:

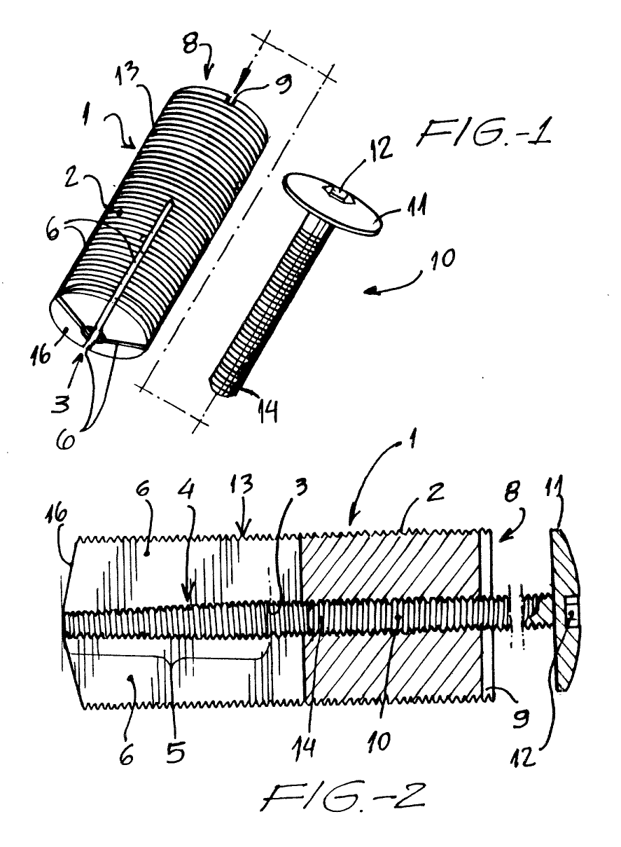

FIG. 1 illustrates a perspective of the screw herein proposed;

FIG. 2 illustrates a full cut view of an initial assembly stage of the screw in question; and

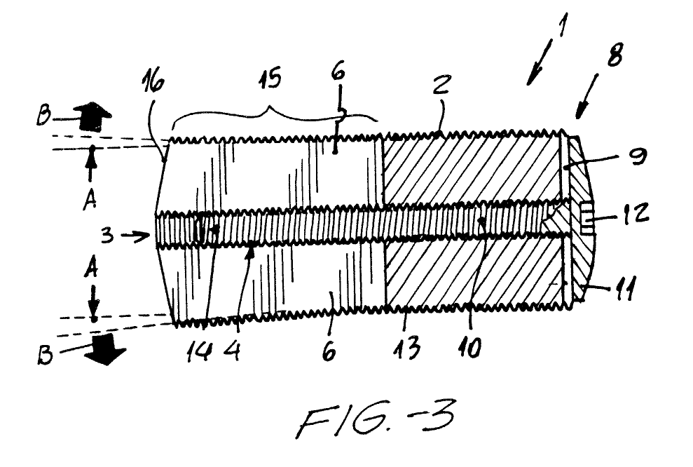

FIG. 3 illustrates a full cut view of the screw herein described, in its assembly final stage.

In conformity with the illustrations in the aforementioned figures, the surgical-use screw herein dealt with is composed by an expandable element 1 formed by a monoblock body 2 with an essentially cylindrical configuration.

The expandable element 1 comprises a threaded axial bore 3, which, on its distal half 4, undergoes a funneling defining an essentially threaded trunk section 5.

The expandable element 1, such as illustrated by FIG. 1, displays a set of slots starting on its distal end and continuing as far as approximately half of the length measurement of the referred element 1.

The proximal end 8 of expandable element 1 incorporates a radial slot 9 which can be employed as a coupling place for an appropriate tool (not illustrated) used to promote the positioning of the mentioned expandable element 1 at its assembly place in the receiving bone structure.

The screw herein dealt with also comprises a spindle 10 which operates as an expanding element of the expandable element 1, and the referred expanding spindle 10 incorporates a head 11, which, at the center of its structural region, incorporates a cavity 12, preferably with a sixangled configuration, destined to be used as coupling place for an appropriate tool (not illustrated) which is employed to promote the rotation and consequent advance of spindle 10 along the threaded axial bore 3.

Externally, the expandable element 1 counts on thread filaments 13 extremely close to one another, and developing themselves along their entire length.

The present screw operation principle can be better understood through FIGS. 2 and 3, and disposes that spindle 10 be assembled in the threaded axial bore 3, after the expandable element 1 had been duly assembled in the receiving bone structure.

The rotation and consequent advance of spindle 10 inside the expandable element 1 does not produces external mechanical effects until the arrival of the mentioned spindle 10 distal end 14 reaches the trunk threaded section 5.

The spindle 10, when advancing against the trunk threaded section 5, progressively promotes the expansion of the distal half 15 of the expandable element 1 (a condition schematically illustrated in FIG. 3 by means of arrows A and B, wherein arrows A indicate the diameter before expansion, and arrows B indicate the expansion movement), thus determining its locking in relation to the bone structure in which the screw is assembled.

As it must have become clear, the configuration herein proposed is more simple and functional than the configuration displayed in document MU 7900887-9, inasmuch as in the proposed surgical-use screw only two parts are employed, that is, the expandable element 1 and the spindle 10.

The distal end of expandable element 1 incorporates a trunk configuration 16 facilitating the introduction of the referred expandable element 1 in the receiving bone structure.

In the configuration herein dealt with, the spindle 10 operates directly in the sense of promoting the expansion of the expandable element 1 structure, not needing accessory elements to promote such operation.

Thus, it is evident that the surgical-use screw herein described displays a substantially more rational configuration when compared to the state-of-the-art.

1. A building configuration introduced in a surgical-use screw characterized by the fact of comprising an expandable element (1) formed by a monoblock body (2) with an essentially cylindrical configuration;
the expandable element (1) comprises a threaded axial bore (3), which at its distal half (4) undergoes a funneling defining a threaded trunk sector (5);
the expandable element (1) displays a set of slots (6), starting from its distal end and reaching as far as approximately half the length measurement of said element (1).

2. A building configuration introduced in a surgical-use screw, according to claim 1, characterized by the fact that the proximal end (8) of the expandable element (1) incorporates a radial indenture (9) which can be employed as a coupling place for an appropriate tool to be used to promote the positioning of the mentioned expandable element (1) at its assembly place in the receiving bone structure.

3. A building configuration introduced in a surgical-use screw, according to claim 1, characterized by the fact that the screw herein dealt with also comprises a spindle (10) operating as an expanding element of the expandable element (1), and the referred expanding spindle (10) incorporates a head (11) which, at the center of its structural region, incorporates a cavity (12), preferably with a sixangled configuration, destined to be used as coupling place for an appropriate tool (not illustrated) which is employed to promote the rotation and consequent advance of spindle (10) along the threaded axial bore (3).

4. A building configuration introduced in a surgical-use screw, according to claim 1, characterized by the fact that, externally, the expandable element (1) counts on thread filaments (13) extremely close to one another, and which develop themselves along their entire length.

5. A building configuration introduced in a surgical-use screw, according to claim 1, characterized by the fact that the expandable element (1) distal end incorporates a trunk configuration (16).
